(19) United States
(12) Patent Application Publication
(10) Pub. No.: US 2022/0330577 A1
(43) Pub. Date: Oct. 20, 2022

(54) A METHOD FOR IMPROVING THE NUTRITIONAL VALUE OF ANIMAL FEED

(71) Applicants: DSM IP ASSETS B.V., Heerlen (NL); NOVOZYMES A/S, Bagsvaerd (DK)

(72) Inventors: Aaron COWIESON, Kaiseraugst (CH); Jose-Otavio SORBARA, Kaiseraugst (CH)

(21) Appl. No.: 17/633,106

(22) PCT Filed: Aug. 5, 2020
(86) PCT No.: PCT/EP2020/071962

§ 371 Date: Feb. 4, 2022

(30) Foreign Application Priority Data
Aug. 6, 2019 (EP) 19190167.7

(51) Int. Cl. A23K 20/189; C12N 9/16; C12N 9/52; A23K 20/147; A23K 10/30; A23K 50/75

(52) U.S. Cl. CPC A23K 20/189; C12Y 301/03026; C12N 9/16; C12N 9/52; A23K 20/147; A23K 10/30; A23K 50/75

(57) ABSTRACT

The invention relates to the use of at least one bacterial phytase in combination with one or more protease(s) in animal feed for improving nutrient and E ileal digestibility of animal feed, in particular an improved digestibility of Threonine, Proline and Cysteine, the method comprising the step of applying to the animal a feed with an efficient amount of one or more proteolytic enzyme in combination with at least one phytase.

The present invention relates to a method for improving the nutritional value of animal feed. More specifically, the invention relates to a method for improving nutrient and E ileal digestibility of animal feed, in particular an improved digestibility of Threonine, Proline and Cysteine, the method comprising the step of applying to the animal a feed with an efficient amount of one or more proteolytic enzyme in combination with at least one phytase.

The invention furthermore relates to an animal feed composition comprising a soybean meal/corn diet mixed with an enzyme composition comprising at least one phytase and one or more proteolytic enzyme, i.e. protease.

It has been found that adding phytase alone to corn or to soybean meal (SBM) had a limited effect on the ileal digestibility of N. Adding protease alone to corn or SBM had a modest benefit. Adding phytase on top of protease to either corn or SBM gave an effect that was larger than the sum of the individual contributions i.e. phytase and protease had a synergistic effect on corn and SBM as individual ingredients. The same pattern of phytase and protease effect can be seen in the mixture of corn and SBM (a blend to approximate a commercial diet). However, here we apparently see a second tier synergy whereby blending enzyme treated corn and SBM together gives a digestibility value that is greater than either individually.

Phytases (myo-inositol hexakisphosphate phosphohydrolases; EC 3.1.3.8) are enzymes that hydrolyze phytate (myo-inositol hexakisphosphate) to myo-inositol and inorganic phosphate and are known to be valuable feed additives.

A variety of Phytases differing in pH optima, substrate specificity, and specificity of hydrolysis have been identified in plants and fungi. Acid Phytases from wheat bran and Aspergilli have been extensively studied and the stereo specificity of hydrolysis has been well established. Based on the specificity of initial hydrolysis, two classes of acid Phytases are recognized by the International Union of Pure and Applied Chemistry and the International Union of Biochemistry (IUPAC-IUB, 1975), the 6-Phytase, found for example in plants, and the 3-Phytase, found in fungi. The 6-Phytase hydrolyses the phosphate ester at the L-6 (or D-4) position of phytic acid, and the 3-Phytase hydrolyses the phosphate ester at the D-3 position.

The ENZYME site at the internet (http://www.expasy.ch/enzyme/) is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch A. The ENZYME database, 2000, Nucleic Acids Res 28:304-305). See also the handbook Enzyme Nomenclature from NC-IUBMB, 1992).

According to the ENZYME site, two different types of phytases are known: A so-called 3-phytase (myo-inositol hexaphosphate 3-phosphohydrolase, EC 3.1.3.8) and a so-called 6-phytase (myo-inositol hexaphosphate 6-phosphohydrolase, EC 3.1.3.26). For the purposes of the present invention, both types are included in the definition of phytase.

Examples of Ascomycete phytases are those derived from a strain of Aspergillus, for example Aspergillus awamori PHYA (SWISSPROT P34753, Gene 133:55-62 (1993)), Aspergillus niger (ficuum) PHYA (SWISSPROT P34752, EP 420358, Gene 127:87-94 (1993)), Aspergillus awamori PHYB (SWISSPROT P34755, Gene 133:55-62 (1993)), Aspergillus niger PHYB (SWISSPROT P34754, Biochem. Biophys. Res. Commun. 195:53-57(1993)); or a strain of Emericella, for example Emericella nidulans PHYB (SWISSPROT O00093, Biochim. Biophys. Acta 1353:217-223 (1997)); or a strain of Thermomyces (Humicola), for example the Thermomyces lanuginosus phytase described in WO 97/35017. Other examples of Ascomycete phytases are disclosed in EP 684313 (for example derived from strains of Aspergillus fumigatus, Aspergillus terreus, and Myceliophthora thermophila); JP 11000164 (a phytase derived from a strain of Penicillium.); U.S. Pat. No. 6,139,902 (a phytase derived from a strain of Aspergillus), and WO 98/13480 (Monascus anka phytase).

Examples of basidiomycete phytases are the phytases derived from Paxillus involutus, Trametes pubescens, Agrocybe pediades and Peniophora lycii (see WO 98/28409).

In the present context, a preferred Phytase according to the invention is classified as belonging to the EC 3.1.3.26 group. The EC numbers refer to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html.

Examples of Phytases for use according to the present inventions are:

Phytases derived from strains of E coli, from strains of Buttiauxella, Ascomycete Phytases as disclosed in EP 684313 (for example derived from strains of Aspergillus fumigatus, Aspergillus terreus, and Myceliophthora thermophila); JP 11000164 (a Phytase derived from a strain of Penicillium.); U.S. Pat. No. 6,139,902 (a Phytase derived from a strain of Aspergillus), WO 98/13480 (Monascus anka Phytase), WO 2008/116878 and WO 2010/034835 (Hafnia phytase).

A preferred Phytase for use according to the invention is derived from a species of E coli, Peniophora, Citrobacter, Hafnia or Buttiauxella.

Examples of Peniophora species are: Peniophora aurantiaca, P. cinerea, P. decorticans, P. duplex, P. ericsonii, P. incamate, P. lycii, P. meridionalis, P. nuda, P. piceae, P. pini, P. pithya, P. polygonia, P. proxima, P. pseudo-pini, P. rufa, P. versicolor, and species simply classified as Peniophora sp. A preferred species is Peniophora lycii. A preferred strain is Peniophora lycii CBS 686.96.

For purposes of the present invention, preferred phytases are the phytases contained in the following commercial products: Ronozyme® HiPhos, Ronozyme® NP and Ronozyme® P (DSM Nutritional Products AG), Natuphos™ (BASF), Finase® and Quantum® Blue (AB Enzymes), OptiPhos® (Huvepharma) Phyzyme® XP (Verenium/DuPont) and Axtra® PHY (DuPont).

For the purposes of the present invention the phytase activity is determined in the unit of FYT, one FYT being the amount of enzyme that liberates 1 micro-mol inorganic ortho-phosphate per min. under the following conditions: pH 5.5; temperature 37° C.; substrate: sodium phytate (C6H6O24P6Na12) in a concentration of 0.0050 mol/l. Suitable phytase assays are the FYT and FTU assays described in Example 1 of WO 00/20569. FTU is for determining phytase activity in feed and premix.

Specific activity is measured on highly purified samples (an SDS poly acryl amide gel should show the presence of only one component). The enzyme protein concentration may be determined by amino acid analysis, and the phytase activity in the units of FYT. Specific activity is a characteristic of the specific phytase variant in question, and it is calculated as the phytase activity measured in FYT units per mg phytase enzyme protein.

For determining mg Phytase protein per kg feed or feed additive, the enzyme is purified from the feed composition or the feed additive, and the specific activity of the purified enzyme is determined using a relevant assay. The Phytase activity of the feed composition or the feed additive is also determined using the same assay, and on the basis of these two determinations, the dosage in mg Phytase protein per kg feed is calculated.

According to the invention, the phytase should of course be applied in an effective amount, i.e. in an amount adequate for improving nutritional value of feed if it is used in combination with a proteolytic enzyme [obtaining the desired effect, e.g. improving FCR]. It is at present contemplated that the phytase is administered in such amounts that the specific activity in the final feed is between 1000 FYT/kg feed and 5000 FYT/kg feed. In particular embodiments, the specific activity is at least 1500, 1700, 1900, 2000, 2100, 2300, 2500, 2700, 2900, 3000, 3100, 3300, 3500, 3700, 3900, 4100, 4300, 4500, 4700, 4900 or 5000 FYT/kg feed.

Proteolytic enzymes or proteases, or peptidases, catabolize peptide bonds in proteins breaking them down into fragments of amino acid chains, or peptides.

Proteases are classified on the basis of their catalytic mechanism into the following groups: serine proteases (S), cysteine proteases (C), aspartic proteases (A), metalloproteases (M), and unknown, or as yet unclassified, proteases (U), see Handbook of Proteolytic Enzymes, A. J. Barrett, N. D. Rawlings, J. F. Woessner (eds), Academic Press (1998), in particular the general introduction part.

Proteases for use according to the invention are acid stable proteases, preferably acid stable serine proteases.

In a particular embodiment, the protease for use according to the invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i. e. the term covers the expression of wild-type, naturally occurring proteases, as well as expression in any host of recombinant, genetically engineered or synthetic proteases.

Examples of microorganisms are bacteria, e. g. bacteria of the phylum Actinobacteria phy. nov., e. g. of class I: Actinobacteria, e. g. of the Subclass V: Actinobacteridae, e. g. of the Order I: Actinomycetales, e. g. of the Suborder XII: Streptosporangineae, e. g. of the Family II: Nocardiopsaceae, e. g. of the Genus I: Nocardiopsis, e. g. Nocardiopsis sp. NRRL 18262, and Nocardiopsis alba; e.g. of the species Bacillus or mutants or variants thereof exhibiting protease activity. This taxonomy is on the basis of Berge's Manual of Systematic Bacteriology, 2nd edition, 2000, Springer (preprint: Road Map to Bergey's).

Preferred proteases according to the invention are acid stable serine proteases obtained or obtainable from the order Actinomycetales, such as those derived from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases.

The term serine protease refers to serine peptidases and their clans as defined in the above Handbook. In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. Examples of serine proteases for use according to the invention are proteases of Clan SA, e. g. Family S2 (Streptogrisin), e. g. Sub-family S2A (alpha-lytic protease), as defined in the above Handbook.

Protease activity can be measured using any assay, in which a substrate is employed, that includes peptide bonds relevant for the specificity of the protease in question. Examples of protease substrates are casein, and pNA-substrates, such as Suc-AAPF-pNA (available e.g. from Sigma S-7388). Another example is Protazyme AK (azurine dyed crosslinked casein prepared as tablets by Megazyme T-PRAK). Example 2 of WO 01/58276 describes suitable protease assays. A preferred assay is the Protazyme assay of Example 2D (the pH and temperature should be adjusted to the protease in question as generally described previously).

There are no limitations on the origin of the acid stable serine protease for use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by Random Mutagenesis. The preparation of consensus proteins is described in e. g. EP 0 897 985.

Examples of acid-stable proteases for use according to the invention are proteases derived from Nocardiopsis sp. NRRL 18262, and Nocardiopsis alba and proteases of at least 60, 65, 70, 75, 80, 85, 90, or at least 95% amino acid identity to any of these proteases.

For calculating percentage identity, any computer program known in the art can be used. Examples of such computer programs are the Clustal V algorithm (Higgins, D. G., and Sharp, P. M. (1989), Gene (Amsterdam), 73, 237-244; and the GAP program provided in the GCG version 8 program package (Program Manual for the Wisconsin Package, Version 8, Genetics Computer Group, 575 Science Drive, Madison, Wis., USA 53711) (Needleman, S. B. and Wunsch, C. D., (1970), Journal of Molecular Biology, 48, 443-453.

In another particular embodiment, the protease for use according to the invention, besides being acid-stable, is also thermostable.

The term thermostable means for proteases one or more of the following: That the temperature optimum is at least 50° C., 52° C., 54° C., 56° C., 58° C., 60° C., 62° C., 64° C., 66° C., ° 68 C, or at least ° 70 C.

A commercially available serine proteases derived from Nocardiopsis is Ronozyme® ProAct® (DSM Nutritional Products AG).

In the use according to the invention it is at present contemplated that the protease is administered in a dosage of between 10′000 units/kg feed and 30′000 units/kg feed, for example in one of the following amounts (dosage ranges): 10′000 units/kg feed, 11′000, 12′000, 13′000, 14′000, 15′000, 16′000, 17′000, 18′000, 19′000, 20′000 units/kg feed. One protease unit (PROT) is the amount of enzyme that releases 1 μmol of p-nitroaniline from 1 mM substrate (Suc-Ala-Ala-Pro-Phe-pnA) per minute at pH 9.0 and 37° C.

In a particular embodiment, the phytase and the protease, in the form in which they are added to the feed, or when being included in a feed additive, are well-defined. Well-defined means, that the enzyme preparation is at least 50% pure on a protein-basis. In other particular embodiments the enzyme preparation is at least 60, 70, 80, 85, 88, 90, 92, 94, or at least 95% pure. Purity may be determined by any method known in the art, e.g. by SDS-PAGE, or by Size-exclusion chromatography (see Example 12 of WO 01/58275).

A well-defined enzyme preparation is advantageous. For instance, it is much easier to dose correctly to the feed an enzyme that is essentially free from interfering or contaminating other enzymes. The term dose correctly refers in particular to the objective of obtaining consistent and constant results, and the capability of optimising dosage based upon the desired effect.

For the present purposes, the term animal includes all animals, including human beings. In a particular embodiment, the phytase variants and compositions of the invention can be used as a feed additive for non-human animals. Examples of animals are non-ruminants, and ruminants, such as cows, sheep and horses. In a particular embodiment, the animal is a non-ruminant animal. Non-ruminant animals include mono-gastric animals, e.g. pigs or swine (including, but not limited to, piglets, growing pigs, and sows); poultry such as turkeys and chicken (including but not limited to broiler chicks, layers); young calves; and fish (including but not limited to salmon).

The term feed or feed composition means any compound, preparation, mixture, or composition suitable for, or intended for intake by an animal. The feed can be fed to the animal before, after, or simultaneously with the diet. The latter is preferred.

The composition of the invention, when intended for addition to animal feed, may be designated an animal feed additive. Such additive always comprises the enzymes in question, preferably in the form of stabilized liquid or dry compositions. The additive may comprise other components or ingredients of animal feed. The so-called pre-mixes for animal feed are particular examples of such animal feed additives. Pre-mixes may contain the enzyme(s) in question, and in addition at least one vitamin and/or at least one mineral.

In a preferred example, the phytase and the protease, which are added to the feed via a feed additive composition, are dosed such that the final feed has the following dosages:

Phytase: at least 2′000 FYT/kg feed and Protease: 15′000 units/kg feed, or
Phytase: 3′000 FYT/kg feed and Protease: 15′000 units/kg feed.

Accordingly, in a particular embodiment, in addition to the component polypeptides, the composition of the invention may comprise or contain at least one fat-soluble vitamin, and/or at least one water-soluble vitamin, and/or at least one trace mineral. Also at least one macro mineral may be included.

Examples of fat-soluble vitamins are vitamin A, D3, E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

Further, optional, feed-additive ingredients are colouring agents, aroma compounds, stabilizers, additional enzymes, and antimicrobial peptides.

Additional enzyme components of the composition of the invention include at least one polypeptide having xylanase activity; and/or at least one polypeptide having endoglucanase activity; and/or at least one polypeptide having endo-1,3(4)-beta-glucanase activity.

Xylanase activity can be measured using any assay, in which a substrate is employed, that includes 1,4-beta-D-xylosidic endo-linkages in xylans. Different types of substrates are available for the determination of xylanase activity e.g. Xylazyme cross-linked arabinoxylan tablets (from MegaZyme), or insoluble powder dispersions and solutions of azo-dyed arabinoxylan.

Endoglucanase activity can be determined using any endoglucanase assay known in the art. For example, various cellulose- or beta-glucan-containing substrates can be applied. An endoglucanase assay may use AZCL-Barley beta-Glucan, or preferably (1) AZCL-HE-Cellulose, or (2) Azo-CM-cellulose as a substrate. In both cases, the degradation of the substrate is followed spectrophotometrically at OD595 (see the Megazyme method for AZCL-polysaccharides for the assay of endo-hydrolases at http://www.megazyme.com/booklets/AZCLPOL.pdf.

Endo-1,3(4)-beta-glucanase activity can be determined using any endo-1,3(4)-beta-glucanase assay known in the art. A preferred substrate for endo-1,3(4)-beta-glucanase activity measurements is a cross-linked azo-coloured beta-glucan Barley substrate, wherein the measurements are based on spectrophotometric determination principles.

For assaying xylanase, endoglucanase, beta-1,3(4)-glucanase and protease activity the assay-pH and the assay-temperature are to be adapted to the enzyme in question (preferably a pH close to the optimum pH, and a temperature close to the optimum temperature). A preferred assay pH is in the range of 2-10, preferably 3-9, more preferably pH 3 or 4 or 5 or 6 or 7 or 8, for example pH 3 or pH 7. A preferred assay temperature is in the range of 20-80° C., preferably 30-80° C., more preferably 40-75° C., even more preferably 40-60° C., preferably 40 or 45 or 50° C. The enzyme activity is defined by reference to appropriate blinds, e.g. a buffer blind.

Examples of antimicrobial peptides (AMP's) are CAP18, Leucocin A, Tritrpticin, Protegrin-1, Thanatin, Lactoferrin, Lactoferricin, and Ovispirin such as Novispirin (Robert Lehrer, 2000), and variants or fragments thereof which retain antimicrobial activity. Other examples are anti-fungal polypeptides (AFP's) such as those derived from Aspergillus giganteus, and Aspergillus niger, as well as variants and fragments thereof which retain antifungal activity, as disclosed in WO 94/01459 and PCT/DK02/00289.

In a particular embodiment, the animal feed additive of the invention is intended for being included (or prescribed as having to be included) in animal diets or feed at levels of 0.0010-12.0%, or 0.0050-11.0%, or 0.0100-10.0%; more particularly 0.05-5.0%; or 0.2-1.0% (% meaning g additive per 100 g feed). This is so in particular for premixes.

Accordingly, the concentrations of the individual components of the animal feed additive, e.g. the premix, can be found by multiplying the final in-feed concentration of the same component by, respectively, 10-10000; 20-2000; or 100-500 (referring to the above three percentage inclusion intervals).

The final in-feed concentrations of important feed components may reflect the nutritional requirements of the animal, which are generally known by the skilled nutritionist, and presented in publications such as the following: NRC, Nutrient requirements in swine, ninth revised edition 1988, subcommittee on swine nutrition, committee on animal nutrition, board of agriculture, national research council. National Academy Press, Washington, D.C. 1988; and NRC, Nutrient requirements of poultry, ninth revised edition 1994, subcommittee on poultry nutrition, committee on animal nutrition, board of agriculture, national research council, National Academy Press, Washington, D.C., 1994.

The composition of the invention can be prepared according to methods known in the art, e.g. by mixing the phytase and the protease with the additional ingredients, if any.

Animal feed compositions or diets have a relatively high content of protein. An animal feed composition according to the invention has a crude protein content of 50-800, or 75-700, or 100-600, or 110-500, or 120-490 g/kg, and furthermore comprises a composition of the invention.

Furthermore, or in the alternative (to the crude protein content indicated above), the animal feed composition of the invention has a content of metabolisable energy of 10-30, or 11-28, or 11-26, or 12-25 MJ/kg; and/or a content of calcium of 0.1-200, or 0.5-150, or 1-100, or 4-50 g/kg; and/or a content of available phosphorus of 0.1-200, or 0.5-150, or 1-100, or 1-50, or 1-25 g/kg; and/or a content of methionine of 0.1-100, or 0.5-75, or 1-50, or 1-30 g/kg; and/or a content of methionine plus cysteine of 0.1-150, or 0.5-125, or 1-80 g/kg; and/or a content of lysine of 0.5-50, or 0.5-40, or 1-30 g/kg.

Crude protein is calculated as nitrogen (N) multiplied by a factor 6.25, i.e. Crude protein (g/kg)=N (g/kg)×6.25 as stated in Animal Nutrition, 4th edition, Chapter 13 (Eds. P. McDonald, R. A. Edwards and J. F. D. Greenhalgh, Longman Scientific and Technical, 1988, ISBN 0-582-40903-9). The nitrogen content can be determined by the Kjeldahl method (A.O.A.C., 1984, Official Methods of Analysis 14th ed., Association of Official Analytical Chemists, Washington D.C.). But also other methods can be used, such as the so-called Dumas method in which the sample is combusted in oxygen and the amount of nitrous gasses formed are analysed and recalculated as nitrogen.

Metabolisable energy can be calculated on the basis of the NRC publication Nutrient Requirements of Swine (1988) pp. 2-6, and the European Table of Energy Values for Poultry Feed-stuffs, Spelderholt centre for poultry research and extension, 7361 DA Beekbergen, The Netherlands. Grafisch bedrijf Ponsen & Iooijen by, Wageningen. ISBN 90-71463-12-5.

In a particular embodiment, the animal feed composition of the invention contains at least one vegetable protein or protein source. Examples of vegetable proteins or protein sources are soybean, peas and rape seed from leguminosae and brassica families, and the cereals such as barley, maize (corn), oat, rice, rye, sorghum and wheat.

In a preferred embodiment of the invention the animal feed composition is formulated to contain an AME of 3050 kcal/kg, 220 g/kg crude protein, 9 g/kg calcium, 4.5 g/kg available phosphorus and 12.3 g/kg digestible lysine.

Animal diets can e.g. be manufactured as mash feed (non-pelleted) or pelleted feed.

Typically, the milled feed-stuffs are mixed and sufficient amounts of essential vitamins and minerals are added according to the specifications for the species in question.

The phytase and protease of the invention can be added in the form of a solid or liquid enzyme formulation, or in the form of a feed additive, such as a pre-mix. A solid composition is typically added before or during the mixing step; and a liquid composition is typically added after the pelleting step.

The phytase and protease of the invention when added to animal feed leads to an improved nutritional value of the feed, e.g. the growth rate and/or the weight gain and/or the feed conversion (i.e. the weight of ingested feed relative to weight gain) of the animal is/are improved.

In particular embodiments the weight gain is at least 101, 102, 103, 104, 105, 106, 107, 108, 109, or at least 110% of the control (no enzyme addition).

In further particular embodiments the feed conversion is at most (or not more than) 99, 98, 97, 96, 95, 94, 93, 92, 91 or at most 90%, as compared to the control (no enzyme addition).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The present invention is further described by the following examples which should not be construed as limiting the scope of the invention.

### EXAMPLE 1: SPECIFIC ACTIVITY OF PHYTASES

The specific activity of phytases can be determined on highly purified samples dialysed against 20 mM sodium acetate, pH 5.5. The purity can be checked beforehand on an SDS poly acryl amide gel showing the presence of only one component.

The protein concentration can be determined by amino acid analysis as follows: An aliquot of the sample is hydrolyzed in 6N HCl, 0.1% phenol for 16 h at 110 C in an evacuated glass tube. The resulting amino acids is quantified using an Applied Biosystems 420A amino acid analysis system operated according to the manufacturer's instructions. From the amounts of the amino acids the total mass—and thus also the concentration—of protein in the hydrolyzed aliquot can be calculated.

The phytase activity is determined in the units of FYT, and the specific activity is calculated as the phytase activity measured in FYT units per mg phytase variant enzyme protein.

### EXAMPLE 2: ANIMAL TRIAL—TOWARD STANDARDIZED AMINO ACID MATRICES FOR EXOGENOUS PHYTASE AND PROTEASE IN CORN/SOY-BASED DIETS FOR BROILERS

Materials and Methods

Birds and Diets

The study procedures were reviewed and approved by the Massey University Animal Ethics Committee and complied with the New Zealand Code of Practice for the Care and Use of Animals for Scientific Purposes.

A total of 468 male Ross 308 broiler chicks were obtained at 1 d of age from a commercial hatchery and reared in an environmentally-controlled room until d 21. A pre-experimental corn/soy-based broiler starter diet that met all the nutrient requirements of the birds was fed from d1 to 21. This diet was formulated to contain an AME of 3050 kcal/kg, 220 g/kg crude protein, 9 g/kg calcium, 4.5 g/kg available phosphorus and 12.3 g/kg digestible lysine. On d 21, birds were randomly distributed to 78 wire-floored metabolism cages (6 birds per cage) and offered one of 13 experimental diets (Table 1; 6 replicate cages per diet) from d21 to 28 or a nitrogen (N)-free diet (d 25-28). The experimental diets were based on the standardized ileal amino acid digestibility assay protocol (Ravindran et al., 2017) and were based on corn, soybean meal or a mixture of corn and soybean meal (Table 1). Each diet was fed without or with phytase (RONOZYME HiPhos, DSM Nutritional Products, Kaiseraugst, Switzerland, 3000 FYT/kg feed), protease (RONOZYME ProAct, DSM Nutritional Products, Kaiseraugst, Switzerland, 15000 PROT/kg feed) ora combination of phytase and protease (at the same inclusion concentration). One protease unit (PROT) is defined as the amount of enzyme that releases 1 mmol of p-nitroaniline from 1 mM substrate (Suc-Ala-Ala-Pro-Phe-pNA) per minute at pH 9.0 and 37° C. One phytase unit (FYT) is defined as the quantity of enzyme which liberates 1 μmol of inorganic phosphate per minute from 5.0 μmol/l sodium phytate at pH 5.5 and 37° C. Birds that received the N-free diet received the commercial starter diet until d25 (Ravindran et al. 2017). The cages were housed in an environmentally-controlled room. Temperature was maintained at 32° C. on d 1 and gradually reduced to 24° C. by d 21 and further to 21° C. by d28. Temperature modification was achieved by the use of thermostatically controlled fans and electric heaters. The birds received 20 hours fluorescent illumination per day and were allowed free access to diets and water. Birds were checked at least 3 times per day (09.00, 13.00 and 16.00 hrs) and any unusual aspect of bird behavior or condition was recorded. Sick or injured animals were weighed and removed from the study. All diets contained titanium dioxide (TiO2; 5 g/kg) as an indigestible marker.

### Measurements

On d28, all birds from each replicate cage were euthanized by intra-cardial injection of sodium pentobarbitone for ileal digesta collection. The small intestine was immediately exposed and the contents of the distal half of the ileum were collected by gently flushing with distilled water into plastic containers. The ileum was defined as that portion of the small intestine extending from vitelline diverticulum to a point 40 mm proximal to the ileo-caecal junction. Digesta from birds within a cage were pooled, resulting in 6 samples per dietary treatment. The digesta samples were frozen immediately after collection, lyophilized and processed. Samples of digesta and diets were analyzed for or amino acids, including methionine and cysteine.

### Chemical Analysis

Amino acids (including proline) were determined by hydrolyzing the samples with 6 N HCl (containing phenol) for 24 h at 110±2° C. in glass tubes sealed under vacuum. Amino acids were detected on a Waters ion-exchange HPLC system, and the chromatograms were integrated by using dedicated software (Millenium, version 3.05.01, Waters, Millipore, Milford, Mass.) with the amino acids identified and quantified by using a standard amino acid mixture (product no. A2908, Sigma, St. Louis, Mo.). The HPLC system consisted of anion-exchange column, two 510 pumps, a Waters 715 ultra-WISP sample processor, a column heater, a post-column reaction coil heater, a ninhydrin pump, and a dual-wavelength detector. Amino acids were eluted by a gradient of pH 3.3 sodium citrate eluent to pH 9.8 sodium borate eluent at a flow rate of 0.4 mL/min and a column temperature of 60° C. Cysteine and methionine were analyzed as cysteic acid and methionine sulfone, respectively, by oxidation with performic acid for 16 h at 0° C. and neutralization with hydrobromic acid before hydrolysis (Ravindran et al., 2008).

### Calculations

The apparent ileal digestibility (AID) of AA was calculated by the following formula using the titanium marker ratio in the diet and ileal digesta.

AID of AA=((AA/Ti)_{d}−(AA/Ti)ᵢ)/(AA/Ti)_{d}

Where, (AA/Ti)d=ratio of amino acid and titanium in diet, and (AA/Ti)i=ratio of amino acid and titanium in ileal digesta.

The basal endogenous AA (EAA) flow at the terminal ileum was calculated as grams lost per kilogram of DM intake (DMI; Moughan et al., 1992).

Basal endogenous AA flow (g/kg DMI)=(AA in ileal digesta (g/kg)×Ti_{d} (g/kg))/Tiᵢ (g/kg)

Where, Tid=titanium in diet and Tii=titanium in ileal digesta.

Apparent digestibility data for N and AA were then converted to standardized digestibility values, using endogenous N and AA values determined from birds fed the N-free diet (Ravindran et al., 2017).

SID=AID+[Basal EAA (g/kg DMI)]/Ing. AA (g/kg DM)

Where, AID=apparent ileal digestibility of the AA, Basal EAA=basal endogenous AA loss and Ing. AA=concentration of the AA in the ingredient.

Apparent digestibility values were standardized using the following basal ileal endogenous flow values (g/kg DM intake), determined by feeding N-free diet: N, 1.206; Met, 0.110; Cys, 0.149; Lys, 0.263; Thr, 0.468; Arg, 0.320; Ile, 0.285; Leu, 0.460; Val, 0.380; His, 0.098; Phe, 0.264; Gly, 0.317; Ser, 0.408; Pro, 0.375; Ala, 0.314; Asp, 0.573, Glu, 0.792 and Tyr, 0.261.

### Results

The proximate, mineral and AA composition of the corn and soybean meal (g/kg as received) is presented in Table 2 and all values are in agreement with expectations, including the recovery of titanium dioxide in the feed samples. Phytase and protease activity recovered in the experimental diets is presented in Table 3 and is in line with expectations for these products.

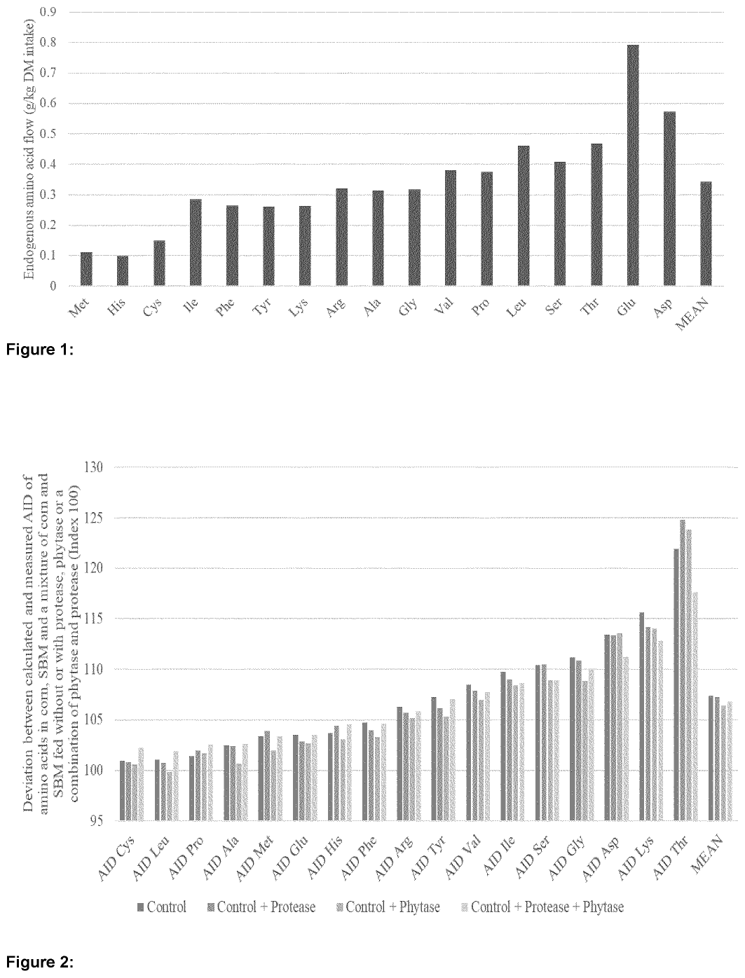

The flow of endogenous AA, presented in the methodology section above, in the terminal ileum of broilers that received the N-free diet are presented in FIG. 1. The mean flow of endogenous AA was 0.34 g/kg DM intake. The flow of endogenous Asp, Glu, Thr, Ser, Leu, Pro and Val were higher than this mean value and other AA (notably Met and His where the endogenous flow was only 0.11 g/kg DM intake) were lower. The flow of endogenous AA in the present experiment (FIG. 4) correlated positively (P<0.001; r²=0.97) with the flow of endogenous AA from broilers fed an N-free diet in previous work.

The AID of AA in corn, SBM, a mixture of corn and SBM and the same without or with phytase, protease or a combination of phytase and protease is presented in Table 4. The AID of all AA other than Met, Cys, Leu and Ala, was higher (P<0.01) in SBM or in the corn/SBM mixture compared with corn. There was no effect (P>0.05) of phytase addition on the AID of AA. However, the effect of protease on the AID of Thr, Pro and Cys was greater when offered in combination with phytase than without, resulting in a significant phytase*protease interaction for those AA. Addition of protease resulted in an increase (P<0.01) in the AID of AA by 3.6%, which ranged from 2.1-2.3% for Arg and Met respectively to 6.0-6.4% for Thr and Cys respectively.

The SID of AA in corn, SBM, a mixture of corn and SBM and the same without or with phytase, protease or a combination of phytase and protease is presented in Table 5. The SID of AA was not significantly different between diets for Glu, Val, Ile, His and Arg whereas the SID of Cys, Met, Phe, Leu, Ala and Pro was higher (P<0.01) in corn compared with SBM. For N and the other AA the SID was generally higher for SBM than for corn. Similar to the AID observations, the presence of phytase increased the effectiveness of protease on the SID of Thr, Pro and Cys, resulting in a significant interaction for those AA. Protease addition resulted in an increase in the SID of all AA by an average of 3.4%, ranging from 2.0% for Arg to 6.1% for Cys.

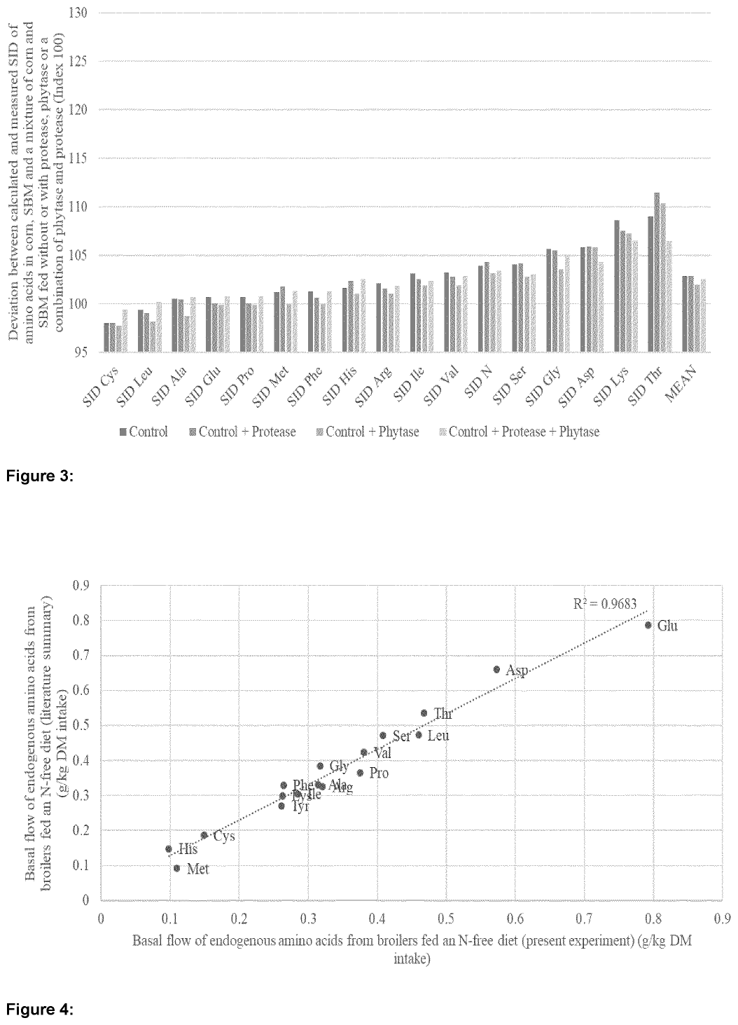

The additivity of AID or SID values and the influence of exogenous enzyme addition on the same is presented in FIGS. 2 and 3. On an AID basis (FIG. 2) the calculated digestibility of the mixture of corn and SBM (based on the determined AID of AA in the individual ingredients and their relative proportions in the mixture i.e. 65% corn and 35% SBM) underestimated the measured digestibility by 7.4%, ranging from 1.0% for Cys to 21.9% for Thr. However, when fed with a combination of phytase and protease this underestimation was reduced (P<0.05) to 6.7%. On a SID basis the calculated digestibility of AA in corn and SBM underestimated the measured digestibility by only 2.9% and the calculated and measured values were not significantly different. Addition of phytase and protease numerically reduced this underestimation to 2.5%, being particularly notable for Thr and Lys.

The effect of diet (corn, SBM or a mixture of corn and SBM) and enzyme (unsupplemented, phytase or protease) on the AID and SID of AA are presented in Tables 4 and 5. Some variance from experiment to experiment is expected due to the influence of the individual ingredient sources used (both corn and SBM are known to vary in digestibility of macro-nutrients; Leeson et al., 1993; Douglas et al., 2000; Ravindran et al., 2007), slight changes in methodology and in the capacity of the birds to extract nutrients from the feed (Hughes & Choct, 1997). Importantly, the corn/SBM mixture has returned a consistently higher AID of AA than the corn or SBM alone. This observation can be observed especially for Thr, Lys, Asp, Gly and Ser where under-estimation of the true value of the individual ingredients was >10% (FIG. 2), further demonstrating the problem of arithmetic assumptions about the nutritional value of individual ingredients in complex diets.

Specifically for Thr, Pro and Cys a significant phytase-protease interaction can be observed. This was caused by the effect of protease being substantially greater when offered in combination with phytase than when either enzyme were fed alone.

### Standardized Ileal Digestibility of Threonine

Control (no enzymes): 80.2%

### Protease: 81.5%

### Phytase: 79.3%

### Protease+Phytase: 86.6%

### Standardized Ileal Digestibility of Proline

Control (no enzymes): 87.2%

### Protease: 88.6%

### Phytase: 87.1%

### Protease+Phytase: 91.1%

### Standardized Ileal Digestibility of Cysteine

Control (no enzymes): 80.3%

### Protease: 83.2%

### Phytase: 80.5%

### Protease+Phytase: 87.4%

The significant interaction between phytase and protease for the AID and SID of Thr, Pro and Cys is intriguing and not easy to explain. In the absence of phytase, the addition of protease increased the SID of Thr, Pro and Cys by 1.6, 1.6 and 3.6% respectively. Phytase alone had no effect on the SID of these AA. However, when protease was added on top of phytase an increase in the SID of Thr (1.6 to 8.0%), Pro (1.6 to 4.5%) and Cys (3.6 to 8.8%) was observed. This statistically-confirmed synergy between phytase and protease is interesting and may be associated with co-operation between these enzymes for access to substrate e.g. protease improving solubility of phytate, or perhaps through physiological effects involving myo-inositol, sodium portioning or, more generally, amino acid absorption and peptide transport.

It can be concluded that the SID AA system for feed ingredient appraisal results in more precise and predictable outcomes for mixed diets than is the case for AID AA approaches and should be used wherever possible. Furthermore, exogenous protease is an effective tool to promote the digestibility of AA in broilers and may do so to a greater extent than is the case for phytase. The synergistic effects of phytase and protease on the SID of Thr, Cys and Pro warrants further attention, especially considering endogenous protein flow, peptide transport and sodium partitioning.

### REFERENCES

Douglas, M. W., C. M. Parsons and M. R. Bedford 2000. Effect of various soybean meal sources and Avizyme on chick growth performance and ileal digestible energy. J. Appl. Poult. Res. 9: 74-80.
Leeson, S., A. Yersin and L. Volker. 1993. Nutritive value of the 1992 corn crop. J. Appl. Poult. Res. 2: 208-213.
Moughan, P. J. and G. S. Marlies Leenaars 1992. Endogenous amino acid flow in the stomach and intestine of the young growing pig. J. Sci. Food. Agric. 60: 437-442.
Moughan, P. J. and S. M. Rutherfurd. 2012. Gut luminal endogenous protein: implications for the determination of ileal amino acid digestibility in humans. Brit. J. Nutr. 108: 258-263.
Ravindran, V. and W. H. Hendriks. 2004. Endogenous amino acid flows at the terminal ileum of broilers, layers and adult roosters. Anim. Sci. 79: 265-271.
Ravindran, V., L. I. Hew, G. Ravindran and W. L. Bryden. 2004. Endogenous amino acid flow in the avian ileum: quantification using three techniques. Brit. J. Nutr. 92: 217-223.
Ravindran, V., L. I. Hew, G. Ravindran and W. L. Bryden. 2007. Apparent ileal digestibility of amino acids in dietary ingredients for broiler chickens. Anim. Sci. 81: 85-97.
Ravindran, V., O. Adeola, M. Rodehutscord, H. Kluth, J. D. van der Klis, E. van Eerden and A. Helmbrecht. 2017. Determination of ileal digestibility of amino acids in raw materials for broiler chickens—results of collaborative studies and assay recommendations. Anim. Feed Sci. Technol. 225:62-72.

### Tables

TABLE 1
Composition¹ (g/kg) of the experimental diets used in
the ileal digestibility assay (21 to 28 d of age).

| | Corn | SBM | Corn/SBM | N-free |
|---|---|---|---|---|
| Ingredient | diet | diet | diet | diet |
| Corn | 930 | — | 600 | — |
| Soybean meal | — | 410 | 330 | — |
| Wheat starch | — | 520 | — | 842 |
| Soybean oil | 30 | 30 | 30 | 50 |
| Sodium bicarbonate | 2.0 | 2.0 | 2.0 | 2.0 |
| Sodium chloride | 2.0 | 2.0 | 2.0 | 2.0 |
| Dicalcium phosphate | 19 | 19 | 19 | 19 |
| Limestone | 10 | 10 | 10 | 10 |
| Titanium dioxide | 5.0 | 5.0 | 5.0 | 5.0 |
| Vitamin premix¹ | 1.0 | 1.0 | 1.0 | 1.0 |
| Trace mineral premix² | 1.0 | 1.0 | 1.0 | 1.0 |
| Solkafloc (Cellulose) | — | — | — | 50 |
| Dipotassium phosphate | — | — | — | 12 |

¹Diets were formulated as per the instructions in Ravindran et al. (2017) to achieve crude protein concentrations of approximately 70 g/kg in the corn diet, 190 g/kg in the SBM diet and 200 g/kg in the corn/SBM mixture.
²Supplied per kilogram of diet: butylated hydroxy toluene, 100 mg; biotin, 0.2 mg; calcium pantothenate, 12.8 mg; cholecalciferol, 60 μg; cyanocobalamin, 0.017 mg; folic acid, 5.2 mg; menadione, 4 mg; niacin, 35 mg; pyridoxine, 10 mg; trans-retinol, 3.33 mg; riboflavin, 12 mg; thiamine, 3.0 mg; dl-α-tocopheryl acetate, 60 mg; choline chloride, 638 mg; Co, 0.3 mg; Cu, 3.0 mg; Fe, 25 mg; I, 1 mg; Mn, 125 mg; Mo, 0.5 mg; Se, 200 μg; Zn, 60 mg (DSM Nutritional Products, Wagga Wagga, NSW, Australia)

TABLE 2
Proximate, mineral and amino acid composition
of corn and soybean meal (g/kg, as received)¹.

| | Corn | Soybean meal |
|---|---|---|
| Dry matter | 917 | 918 |
| Ash | 13.1 | 70.2 |
| Nitrogen | 11.5 | 77.1 |
| Crude Protein (N × 6.25) | 71.9 | 482 |
| Starch | 631 | 17.4 |
| Fat | 38.0 | 18.1 |
| Calcium | 0.86 | 2.90 |
| Total Phosphorus (P) | 2.34 | 6.26 |
| Phytate P | 1.84 | 3.33 |
| Non-phytate P | 0.50 | 2.93 |
| Magnesium | 0.92 | 3.2 |
| Potassium | 3.7 | 26.0 |
| Sodium | 0.046 | 0.13 |
| Iron | 0.023 | 0.10 |
| Chloride | 0.47 | 0.17 |
| Zinc | 0.021 | 0.043 |
| Arginine | 3.76 | 36.75 |
| Histidine | 1.95 | 12.23 |
| Isoleucine | 2.30 | 21.06 |
| Leucine | 7.87 | 35.17 |
| Lysine | 2.47 | 29.82 |
| Methionine | 1.54 | 6.55 |
| Phenylalanine | 3.32 | 23.79 |
| Threonine | 2.43 | 20.17 |
| Valine | 3.41 | 23.21 |
| Alanine | 4.82 | 21.09 |
| Aspartic acid | 4.85 | 59.11 |
| Cysteine | 1.57 | 6.50 |
| Glycine | 3.04 | 21.69 |
| Glutamic acid | 11.72 | 84.41 |
| Proline | 6.37 | 23.77 |
| Serine | 3.35 | 27.21 |
| Tyrosine | 2.99 | 21.63 |

¹Analyses were done in duplicate (AOAC, 2012).

TABLE 3
Expected and measured enzyme activities in samples of the experimental diets

| | RONOZYME | RONOZYME | Phytase | | Protease | |
|---|---|---|---|---|---|---|
| Diet | HiPhos GT¹ | ProAct CT² | Expected³ | Measured | Expected⁴ | Measured |
| Corn | 0 | 0 | 0 | LOD | 0 | LOD |
| Corn | 0 | 0 | 0 | LOD | 0 | LOD |
| Corn | 3000 | 0 | >3000 | 4266 | 0 | LOD |
| Corn | 3000 | 0 | >3000 | 4676 | 0 | LOD |
| SBM | 0 | 15000 | 0 | LOD | 15000 | 16440 |
| SBM | 0 | 15000 | 0 | LOD | 15000 | 18490 |
| SBM | 3000 | 15000 | >3000 | 4970 | 15000 | 14330 |
| SBM | 3000 | 15000 | >3000 | 4144 | 15000 | 14900 |
| Corn/SBM | 0 | 0 | 0 | LOD | 0 | LOD |
| Corn/SBM | 3000 | 0 | >3000 | 4139 | 0 | LOD |
| Corn/SBM | 0 | 15000 | 0 | LOD | 15000 | 14150 |
| Corn/SBM | 3000 | 15000 | >3000 | 4679 | 15000 | 15980 |
| N-free | 0 | 0 | 0 | LOD | 0 | LOD |

All samples were measured in two replicates.
LOD—Limit of detection.
¹Ronozyme ® HiPhos GT inclusion rate was 0.3 g/kg
²Ronozyme ® ProAct CT inclusion rate was 0.2 g/kg.
³Expected enzyme activity due to addition of Ronozyme ® HiPhos.
⁴Expected enzyme activity due to addition of Ronozyme ® ProAct CT.

TABLE 4
Effect of exogenous enzymes on the apparent ileal amino acid digestibility (%) of corn, soybean
meal (SBM) or a mixture of corn and SBM for growing broiler chickens (measured on d 28)

| Diet | Phy | Pro | N | Asp | Thr | Ser | Glu | Pro | Gly | Ala | Val |
|---|---|---|---|---|---|---|---|---|---|---|---|
| Corn | 0 | 0 | 72.9 | 67.9 | 57.3 | 72.9 | 84.7 | 83.9 | 68.9 | 83.0 | 74.4 |
| Corn | 3000 | 0 | 72.4 | 66.5 | 54.2 | 73.0 | 84.7 | 82.8 | 69.3 | 83.2 | 74.0 |
| Corn | 0 | 15000 | 73.9 | 68.8 | 56.2 | 73.9 | 86.1 | 84.3 | 71.0 | 84.4 | 76.0 |
| Corn | 3000 | 15000 | 78.7 | 75.1 | 66.6 | 79.0 | 88.1 | 87.7 | 75.9 | 87.1 | 80.0 |
| SBM | 0 | 0 | 82.9 | 84.3 | 79.3 | 85.9 | 88.4 | 83.4 | 81.7 | 83.7 | 82.6 |
| SBM | 3000 | 0 | 83.6 | 85.2 | 80.4 | 86.5 | 89.3 | 84.2 | 82.5 | 84.6 | 83.8 |
| SBM | 0 | 15000 | 85.3 | 86.7 | 82.1 | 87.9 | 90.0 | 85.5 | 83.7 | 85.3 | 84.7 |
| SBM | 3000 | 15000 | 86.4 | 88.1 | 83.4 | 88.8 | 90.8 | 86.5 | 85.0 | 86.5 | 85.8 |
| Corn/SBM | 0 | 0 | 83.2 | 83.6 | 79.2 | 85.5 | 89.0 | 84.9 | 81.5 | 85.3 | 83.8 |
| Corn/SBM | 3000 | 0 | 82.5 | 82.9 | 78.5 | 84.7 | 88.7 | 84.7 | 80.5 | 84.2 | 82.8 |
| Corn/SBM | 0 | 15000 | 85.1 | 85.1 | 81.4 | 87.0 | 90.0 | 86.4 | 83.7 | 86.7 | 85.3 |
| Corn/SBM | 3000 | 15000 | 88.0 | 88.6 | 85.2 | 89.7 | 92.2 | 89.5 | 87.0 | 89.2 | 88.4 |
| Pooled SEM | | | 1.64 | 2.18 | 2.46 | 1.65 | 1.33 | 1.16 | 1.93 | 1.50 | 1.85 |
| Model P < | | | 0.001 | 0.001 | 0.001 | 0.001 | 0.001 | 0.001 | 0.001 | 0.001 | 0.001 |
| Main Effects | | | | | | | | | | | |
| Corn | | | 74.5^{a} | 69.6^{a} | 58.6^{a} | 74.7^{a} | 85.9^{a} | 84.7 | 71.3^{a} | 84.4 | 76.1^{a} |
| SBM | | | 84.6^{b} | 86.1^{b} | 81.2^{b} | 87.3^{b} | 89.7^{b} | 84.9 | 83.2^{b} | 85.0 | 84.2^{b} |
| Corn/SBM | | | 84.7^{b} | 85.0^{b} | 81.1^{b} | 86.7^{b} | 90.0^{b} | 86.4 | 83.2^{b} | 86.4 | 85.1^{b} |
| P < | | | 0.001 | 0.001 | 0.001 | 0.001 | 0.001 | NS | 0.001 | NS | 0.001 |
| | 0 | | 80.6 | 79.4 | 72.6 | 82.2 | 88.0 | 84.7 | 78.4 | 84.7 | 81.1 |
| | 3000 | | 81.9 | 81.1 | 74.7 | 83.6 | 89.0 | 85.9 | 80.0 | 85.8 | 82.5 |
| | P < | | NS | NS | NS | NS | NS | NS | NS | NS | NS |
| | | 0 | 79.6 | 78.4 | 71.5 | 81.4 | 87.5 | 84.0 | 77.4 | 84.0 | 80.2 |
| | | 15000 | 82.9 | 82.1 | 75.8 | 84.4 | 89.5 | 86.7 | 81.1 | 86.5 | 83.4 |
| | | P < | 0.01 | 0.01 | 0.01 | 0.01 | 0.01 | 0.001 | 0.01 | 0.01 | 0.01 |
| Interaction | | | | | | | | | | | |
| Terms | | | | | | | | | | | |
| Diet*Pro- | | | NS | NS | NS | NS | NS | NS | NS | NS | NS |
| tease P < | | | | | | | | | | | |
| Diet*Phy- | | | NS | NS | NS | NS | NS | NS | NS | NS | NS |
| tase P < | | | | | | | | | | | |
| Phytase*Pro- | | | NS | NS | 0.05 | NS | NS | 0.05 | NS | NS | NS |
| tease P < | | | | | | | | | | | |
| Diet*Phy- | | | NS | NS | NS | NS | NS | NS | NS | NS | NS |
| tase*Pro- | | | | | | | | | | | |
| tease P < | | | | | | | | | | | |

| Diet | Ile | Leu | Tyr | Phe | His | Lys | Arg | Cys | Me [symbol] |
|---|---|---|---|---|---|---|---|---|---|
| Corn | 74.0 | 86.3 | 79.8 | 81.8 | 82.9 | 68.6 | 82.9 | 75.6 | 84 [symbol] |
| Corn | 73.8 | 86.4 | 80.4 | 82.1 | 82.5 | 69.5 | 83.3 | 74.9 | 84 [symbol] |
| Corn | 75.8 | 87.7 | 81.7 | 83.6 | 83.3 | 71.3 | 84.5 | 77.0 | 85 [symbol] |
| Corn | 79.8 | 89.5 | 83.8 | 86.1 | 86.1 | 75.5 | 86.6 | 82.4 | 88.4 |
| SBM | 84.4 | 84.2 | 88.7 | 86.1 | 87.1 | 87.0 | 91.0 | 74.6 | 86.2 |
| SBM | 85.7 | 85.3 | 89.3 | 87.2 | 87.7 | 87.8 | 91.9 | 75.8 | 86.9 |
| SBM | 86.3 | 86.1 | 89.9 | 88.0 | 88.6 | 88.7 | 92.3 | 79.3 | 87.3 |
| SBM | 87.4 | 87.2 | 90.4 | 88.8 | 89.6 | 89.5 | 92.7 | 81.0 | 88.4 |
| Corn/SBM | 85.2 | 86.5 | 88.9 | 87.3 | 87.5 | 86.8 | 91.1 | 76.0 | 88.2 |
| Corn/SBM | 84.5 | 85.9 | 88.0 | 86.7 | 86.9 | 86.5 | 90.8 | 75.7 | 87.2 |
| Corn/SBM | 86.6 | 87.8 | 89.7 | 88.5 | 88.8 | 88.4 | 92.2 | 78.4 | 89.4 |
| Corn/SBM | 89.5 | 90.4 | 92.1 | 91.1 | 91.3 | 90.7 | 93.9 | 83.7 | 91.4 |
| Pooled SEM | 1.96 | 1.26 | 1.48 | 1.36 | 1.26 | 2.81 | 1.41 | 1.58 | 1.65 |
| Model P < | 0.001 | 0.001 | 0.001 | 0.001 | 0.001 | 0.001 | 0.001 | 0.001 | 0.001 |
| Main Effects | | | | | | | | | |
| Corn | 75.8^{a} | 87.5 | 81.4^{a} | 83.4^{a} | 83.7^{a} | 71.2^{a} | 84.3^{a} | 77.5 | 85.8^{a} |
| SBM | 86.0^{b} | 85.7 | 89.6^{b} | 87.5^{b} | 88.2^{b} | 88.2^{b} | 92.0^{b} | 77.7 | 87.2^{ab} |
| Corn/SBM | 86.5^{b} | 87.6 | 89.7^{b} | 88.4^{b} | 88.6^{b} | 88.1^{b} | 92.0^{b} | 78.5 | 89.0^{b} |
| P < | 0.001 | NS | 0.001 | 0.001 | 0.001 | 0.001 | 0.001 | NS | 0.05 |
| | 82.1 | 86.4 | 86.5 | 85.9 | 86.4 | 81.8 | 89.0 | 76.8 | 86.9 |
| | 83.4 | 87.5 | 87.4 | 87.0 | 87.3 | 83.2 | 89.9 | 78.9 | 87.8 |
| | NS | NS | NS | NS | NS | NS | NS | 0.05 | NS |
| | 81.3 | 85.8 | 85.9 | 85.2 | 85.7 | 81.0 | 88.5 | 75.4 | 86.4 |
| | 84.2 | 88.1 | 87.9 | 87.7 | 87.9 | 84.0 | 90.4 | 80.3 | 88 [symbol] |
| | 0.05 | 0.01 | 0.05 | 0.01 | 0.01 | 0.05 | 0.05 | 0.001 | 0 [symbol] |
| Interaction | | | | | | | | | |
| Terms | | | | | | | | | |
| Diet*Pro- | NS | NS | NS | NS | NS | NS | NS | NS | NS |
| tease P < | | | | | | | | | |
| Diet*Phy- | NS | NS | NS | NS | NS | NS | NS | NS | NS |
| tase P < | | | | | | | | | |
| Phytase*Pro- | NS | NS | NS | NS | NS | NS | NS | 0.05 | NS |
| tease P < | | | | | | | | | |
| Diet*Phy- | NS | NS | NS | NS | NS | NS | NS | NS | NS |
| tase*Pro- | | | | | | | | | |
| tease P < | | | | | | | | | |

Values in columns without a common superscript differ significantly (P < 0.05, unless otherwise stated). [symbol]
indicates data missing or illegible when filed

TABLE 5
Effect of exogenous enzymes on the standardized ileal amino acid digestibility (%) of corn, soybean
meal (SBM) or a mixture of corn and SBM for growing broiler chickens (measured on d 28)

| Diet | Phy | Pro | N | Asp | Thr | Ser | Glu | Pro | Gly | Ala | Val |
|---|---|---|---|---|---|---|---|---|---|---|---|
| Corn | 0 | 0 | 82.5 | 78.8 | 74.9 | 84.1 | 90.9 | 89.3 | 78.4 | 88.9 | 84.6 |
| Corn | 3000 | 0 | 82.0 | 77.3 | 71.2 | 84.2 | 90.9 | 88.2 | 78.9 | 89.1 | 84.2 |
| Corn | 0 | 15000 | 83.5 | 79.7 | 73.9 | 85.0 | 92.3 | 89.7 | 80.6 | 90.3 | 86.2 |
| Corn | 3000 | 15000 | 88.3 | 85.9 | 84.2 | 90.1 | 94.3 | 93.1 | 85.5 | 93.1 | 90.2 |
| SBM | 0 | 0 | 84.4 | 85.2 | 81.4 | 87.3 | 89.3 | 84.8 | 83.0 | 85.1 | 84.1 |
| SBM | 3000 | 0 | 85.0 | 86.1 | 82.5 | 87.9 | 90.2 | 85.6 | 83.8 | 85.9 | 85.3 |
| SBM | 0 | 15000 | 86.7 | 87.6 | 84.2 | 89.2 | 90.9 | 87.0 | 85.1 | 86.6 | 86.2 |
| SBM | 3000 | 15000 | 87.9 | 89.0 | 85.5 | 90.1 | 91.7 | 88.0 | 86.3 | 87.9 | 87.3 |
| Corn/SBM | 0 | 0 | 86.4 | 85.8 | 84.1 | 88.7 | 91.0 | 87.6 | 84.5 | 88.0 | 87.2 |
| Corn/SBM | 3000 | 0 | 85.7 | 85.1 | 83.4 | 87.8 | 90.6 | 87.4 | 83.5 | 87.0 | 86.2 |
| Corn/SBM | 0 | 15000 | 88.3 | 87.3 | 86.3 | 90.2 | 91.9 | 89.2 | 86.7 | 89.5 | 88.6 |
| Corn/SBM | 3000 | 15000 | 91.2 | 90.8 | 90.2 | 92.9 | 94.1 | 92.3 | 90.1 | 91.9 | 91.7 |
| Pooled SEM | | | 1.64 | 2.18 | 2.46 | 1.65 | 1.33 | 1.16 | 1.93 | 1.50 | 1.84 |
| Model P < | | | 0.001 | 0.001 | 0.001 | 0.001 | 0.001 | 0.001 | 0.001 | 0.001 | 0.001 |
| Main Effects | | | | | | | | | | | |
| Corn | | | 84.1^{a} | 80.4^{a} | 76.2^{a} | 85.8^{a} | 92.1 | 90.1^{b} | 80.8^{a} | 90.4^{b} | 86.3 |
| SBM | | | 86.0^{b} | 87.0^{b} | 83.4^{b} | 88.6^{b} | 90.5 | 86.4^{a} | 84.5^{b} | 86.4^{a} | 85.7 |
| Corn/SBM | | | 87.9^{b} | 87.2^{b} | 86.0^{b} | 89.9^{b} | 91.9 | 89.1^{b} | 86.2^{b} | 89.1^{b} | 88.4 |
| P < | | | 0.01 | 0.001 | 0.001 | 0.01 | NS | 0.001 | 0.001 | 0.01 | NS |
| | 0 | | 85.3 | 84.0 | 80.8 | 87.4 | 91.0 | 87.9 | 83.0 | 88.1 | 86.1 |
| | 3000 | | 86.7 | 85.7 | 83.0 | 88.8 | 92.0 | 89.1 | 84.7 | 89.2 | 87.5 |
| | P < | | NS | NS | NS | NS | NS | 0.05 | NS | NS | NS |
| | | 0 | 84.3 | 83.0 | 79.7 | 86.7 | 90.5 | 87.2 | 82.0 | 87.3 | 85.3 |
| | | 15000 | 87.6 | 86.7 | 84.1 | 89.6 | 92.5 | 89.9 | 85.7 | 89.9 | 88.4 |
| | | P < | 0.001 | 0.01 | 0.01 | 0.01 | 0.01 | 0.001 | 0.01 | 0.01 | 0.01 |
| Interaction | | | | | | | | | | | |
| Terms | | | | | | | | | | | |
| Diet*Pro- | | | NS | NS | NS | NS | NS | NS | NS | NS | NS |
| tease P < | | | | | | | | | | | |
| Diet*Phy- | | | NS | NS | NS | NS | NS | NS | NS | NS | NS |
| tase P < | | | | | | | | | | | |
| Phytase*Pro- | | | NS | NS | 0.05 | NS | NS | 0.05 | NS | NS | NS |
| tease P < | | | | | | | | | | | |
| Diet*Phy- | | | NS | NS | NS | NS | NS | NS | NS | NS | NS |
| tase*Pro- | | | | | | | | | | | |
| tease P | | | | | | | | | | | |

| Diet | Ile | Leu | Tyr | Phe | His | Lys | Arg | Cys | Me [symbol] |
|---|---|---|---|---|---|---|---|---|---|
| Corn | 85.3 | 91.6 | 87.8 | 89.1 | 87.5 | 78.4 | 90.7 | 84.3 | 91 [symbol] |
| Corn | 85.2 | 91.7 | 88.4 | 89.4 | 87.1 | 79.2 | 91.1 | 83.7 | 91 [symbol] |
| Corn | 87.2 | 93.0 | 89.7 | 90.9 | 87.9 | 81.1 | 92.3 | 85.7 | 91 [symbol] |
| Corn | 91.1 | 94.9 | 91.8 | 93.4 | 90.7 | 85.2 | 94.4 | 91.1 | 94.9 |
| SBM | 85.7 | 85.4 | 89.8 | 87.1 | 87.8 | 87.9 | 91.8 | 76.7 | 87.8 |
| SBM | 87.0 | 86.5 | 90.5 | 88.2 | 88.4 | 88.6 | 92.7 | 77.9 | 88.5 |
| SBM | 87.6 | 87.3 | 91.0 | 89.0 | 89.3 | 89.5 | 93.1 | 81.4 | 88.9 |
| SBM | 88.6 | 88.4 | 91.5 | 89.8 | 90.3 | 90.3 | 93.5 | 83.1 | 90.0 |
| Corn/SBM | 88.1 | 88.9 | 91.4 | 89.5 | 89.1 | 88.7 | 93.0 | 80.1 | 91.3 |
| Corn/SBM | 87.4 | 88.3 | 90.5 | 89.0 | 88.5 | 88.5 | 92.7 | 79.8 | 90.3 |
| Corn/SBM | 89.5 | 90.2 | 92.2 | 90.8 | 90.5 | 90.4 | 94.1 | 82.6 | 92.4 |
| Corn/SBM | 92.4 | 92.8 | 94.6 | 93.4 | 92.9 | 92.7 | 95.8 | 87.9 | 94.4 |
| Pooled SEM | 1.96 | 1.26 | 1.48 | 1.36 | 1.26 | 2.81 | 1.41 | 1.58 | 1.65 |
| Model P < | 0.001 | 0.001 | 0.001 | 0.001 | 0.001 | 0.001 | 0.001 | 0.001 | 0.001 |
| Main Effects | | | | | | | | | |
| Corn | 87.2 | 92.8^{c} | 89.4^{a} | 90.7^{b} | 88.3 | 81.0^{a} | 92.1 | 86.2^{c} | 92.4^{b} |
| SBM | 87.2 | 87.0^{a} | 90.7^{ab} | 88.5^{a} | 90.2 | 89.0^{b} | 92.8 | 79.8^{a} | 88.8^{a} |
| Corn/SBM | 89.4 | 90.0^{b} | 92.2^{b} | 90.7^{b} | 89.0 | 90.1^{b} | 92.1 | 82.6^{b} | 92.1^{b} |
| P < | NS | 0.001 | 0.05 | 0.05 | NS | 0.001 | NS | 0.001 | 0.01 |
| | 87.2 | 89.4 | 90.3 | 89.4 | 88.7 | 86.0 | 82.5 | 81.8 | 90.6 |
| | 88.6 | 90.4 | 91.2 | 90.5 | 89.7 | 87.4 | 93.4 | 83.9 | 91.6 |
| | NS | NS | NS | NS | NS | NS | NS | 0.05 | NS |
| | 86.5 | 88.7 | 89.7 | 88.7 | 88.1 | 85.2 | 92.0 | 80.4 | 90.1 |
| | 89.4 | 91.1 | 91.8 | 91.2 | 90.2 | 88.2 | 93.9 | 85.3 | 92 [symbol] |
| | 0.05 | 0.01 | 0.05 | 0.01 | 0.01 | 0.05 | 0.05 | 0.001 | 0 [symbol] |
| Interaction | | | | | | | | | |
| Terms | | | | | | | | | |
| Diet*Pro- | NS | NS | NS | NS | NS | NS | NS | NS | NS |
| tease P < | | | | | | | | | |
| Diet*Phy- | NS | NS | NS | NS | NS | NS | NS | NS | NS |
| tase P < | | | | | | | | | |
| Phytase*Pro- | NS | NS | NS | NS | NS | NS | NS | 0.05 | NS |
| tease P < | | | | | | | | | |
| Diet*Phy- | NS | NS | NS | NS | NS | NS | NS | NS | NS |
| tase*Pro- | | | | | | | | | |
| tease P | | | | | | | | | |

Values in columns without a common superscript differ significantly (P < 0.05, unless otherwise stated). [symbol]
indicates data missing or illegible when filed

1. A method for increasing nutrient and E ileal digestibility of animal feed in farm animals, the method comprising the step of applying to the animal a feed with an efficient amount of one or more proteolytic enzyme in combination with at least one phytase.

2. The method of claim 1 for increasing the digestibility of Threonine, Proline and Cysteine available in the protein source of animal feed.

3. The method of claim 1, wherein the animal feed comprises a corn/soybean meal diet.

4. The method of claim 1, wherein
a. the phytase is administered in such amounts that the specific activity in the final feed is between 1000 FYT/kg feed and 54000 FYT/kg feed and
b. the protease is administered in a dosage of between 10′000 units/kg feed and 30′000 units/kg feed.

5. The method according to claim 1, wherein the phytase is classified as belonging to the EC 3.1.3.26 group.

6. The method according to claim 1, wherein the protease is an acid stable serine proteases obtained or obtainable from the order Actinomycetales.

7. The method according to claim 6, wherein the protease is an acid stable serine protease derived from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases.

8. Use of one or more proteolytic enzyme in combination with at least one phytase in animal feed for increasing nutrient and E ileal digestibility of animal feed in farm animals.

9. Use according to claim 8 for increasing the digestibility of Threonine, Proline and Cysteine available in the protein source of animal feed.

10. Use according to claim 8, wherein the animal feed comprises a corn/soybean meal diet.

11. Use according to claim 8, wherein
a. the phytase is administered in such amounts that the specific activity in the final feed is between 1000 FYT/kg feed and 5000 FYT/kg feed and
b. the protease is administered in a dosage of between 10′000 units/kg feed and 30′000 units/kg feed.

12. Use according to claim 9, wherein the phytase is classified as belonging to the EC 3.1.3.26 group.

13. Use according to claim 9, wherein the protease is an acid stable serine proteases obtained or obtainable from the order Actinomycetales.

14. The use according to claim 13, wherein the protease is an acid stable serine protease derived from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases.
